(19) United States
(12) Patent Application Publication
(10) Pub. No.: US 2025/0344976 A1
(43) Pub. Date: Nov. 13, 2025

(54) DRIVER ASSISTANCE SYSTEM AND DRIVER ASSISTANCE METHOD

(71) Applicant: HYUNDAI MOBIS CO., LTD., Seoul (KR)

(72) Inventor: Hyun Jun LIM, Yongin-si (KR)

(73) Assignee: HYUNDAI MOBIS CO., LTD., Seoul (KR)

(21) Appl. No.: 19/034,111

(22) Filed: Jan. 22, 2025

(30) Foreign Application Priority Data
May 8, 2024 (KR) 10-2024-0060563

(51) Int. Cl. A61B 5/18; A61B 5/00; B60Q 9/00; G07C 5/00

(52) U.S. Cl. CPC A61B 5/18; A61B 5/746; B60Q 9/00; G07C 5/008

(57) ABSTRACT

A driver assistance system, a method therefor, and a driver assistance system for a vehicle are provided. The driver assistance system includes a wearable device to measure an electroencephalogram (EEG) signal and head movement of a driver of a moving device, a mobile terminal including an application to receive the EEG signal measured by the wearable device, receive information about the head movement measured by the wearable device, receive a traveling information signal acquired by the moving device, process and provide to a server the received EEG signal, the received information about the head movement, and the received traveling information signal, and receive big data-based inattentiveness alarm information from the server based on information provided to the server by the mobile terminal, and a control module to provide an inattentiveness-related alarm to the driver, and control the moving device based on the inattentiveness alarm information.

### CROSS-REFERENCE TO RELATED APPLICATION(S)

Pursuant to 35 U.S.C. § 119(a), this application claims the benefit of an earlier filing date and right of priority to Korean Application No. 10-2024-0060563, filed on May 8, 2024, in the Korean Intellectual Property Office, the entire contents of which are hereby incorporated by reference for all purposes.

### BACKGROUND

### 1. Field

The following description relates to a driver assistance system and a driver assistance method using an electroencephalogram that provide objectivity in determining inattentiveness of a driver through an electroencephalogram signal of the driver and information about head movement of the driver and provide an alarm to the driver upon determining that the driver is in an inattentive state.

### 2. Description of the Related Art

In general, in modern society, the number of casualties resulted from various traffic accidents is increasing along with an explosive increase in vehicles caused by an increase in income and a pursuit of convenience of consumers. To minimize such casualties in the traffic accidents, many countries around the world are making great efforts to develop safety vehicles.

Such a safety vehicle refers to a vehicle that reduces fatalities in the event of the traffic accident, reduces fatigue of a driver, and is convenient to drive. A basic concept thereof is to prevent the accidents in advance to improve safety of the vehicle and protect a pedestrian.

Recently, to provide a more convenient interface to a user, a development of a human-friendly interface using a voice, a facial expression, a gesture, and a bio-signal such as an electroencephalogram, an electrooculogram, and an electromyogram has been attempted.

The electroencephalogram, the representative bio-signal, refers to a bio-signal that directly or indirectly reflects a person's conscious or unconscious state, and refers to a wave that is measured in all areas of a human scalp and mainly has a frequency equal to or lower than 50 Hz with a potential difference of several tens of microvolts.

Such an electroencephalogram may be classified into a delta wave, a theta wave, an alpha wave, a beta wave, a gamma wave, and the like based on the frequency of the wave.

The delta wave is an electroencephalogram with a frequency equal to or lower than 4 Hz and typically appears in a normal sleep state. The theta wave is an electroencephalogram with a frequency in a range from about 4 to 8 Hz, appears mainly when mentally unstable or distracted, and occurs when in a drowsy state or falling asleep. The alpha wave is an electroencephalogram with a frequency in a range from about 8 to 12 Hz and mostly appears clearly when mentally stable and in a comfortable psychological state with eyes closed. The beta wave refers to an electroencephalogram with a frequency in a range from about 12 to 30 Hz, appears stronger when under stress, such as a little anxiety or tension, and mainly appears when paying more than a certain amount of attention. In addition, the gamma wave refers to an electroencephalogram with a frequency in a range from about 30 to 50 Hz and appears in states of extreme arousal and excitement.

As described above, the driver is required to have considerable concentration while driving the vehicle, and the accident resulting in damage to the vehicle and a human life occurs because of momentary carelessness of the driver during the driving. In addition, the carelessness of the driver causes a large-scale accident that not only damages himself, but also damages others.

In addition, because a frequency band, a spectral density, or the like of an electroencephalogram signal measured from the driver may be different for each driver, there is a limit in consistently determining a careless state of the driver via the electroencephalogram signal.

### SUMMARY

The present disclosure is intended to solve the above-described problems, and according to embodiments, the present disclosure provides a driver assistance system and a driver assistance method capable of providing accuracy in determining inattentiveness of a driver based on an electroencephalogram signal of the driver and information about head movement of the driver.

In addition, the present disclosure provides a wearable device with improved performance and light weight by complementing the shortcomings of wearable devices used for electroencephalogram measurement.

The objects to be achieved by the present disclosure are not limited to what has been particularly described hereinabove and other objects not described herein will be more clearly understood by persons skilled in the art from the following detailed description.

In a general aspect of the disclosure, a driver assistance system, includes: a wearable device configured to measure an electroencephalogram (EEG) signal and head movement of a driver of a moving device; a mobile terminal including an application configured to: receive the EEG signal measured by the wearable device; receive information about the head movement measured by the wearable device; receive a traveling information signal acquired by the moving device; process and provide to a server the received EEG signal, the received information about the head movement, and the received traveling information signal; and receive big data-based inattentiveness alarm information from the server based on information provided to the server by the mobile terminal; and a control module configured to: provide an inattentiveness-related alarm to the driver; and control the moving device based on the inattentiveness alarm information.

The inattentiveness alarm information may include at least one of information determined based on at least one of traveling information obtained by a plurality of moving devices, the EEG signal for each piece of the traveling information, the information about the head movement for each piece of the traveling information, or any combination thereof.

The information about the head movement may include at least one of a difference between a head angle of the driver and a reference angle, the number of changes in the head angle of the driver, a duration of a changed head angle of the driver, or any combination thereof.

The inattentiveness alarm information may include at least one of information about a posture of the driver, information about a drowsy state of the driver, or any combination thereof.

The control module may be further configured to provide at least one of the inattentiveness-related alarm to the driver according to one or more of the posture, the drowsy state related to the inattentiveness alarm information, or any combination thereof.

The application may be further configured to: measure a correct posture characteristic of the driver using the information about the head movement measured during a predetermined time period while the driver wears the wearable device and drives the moving device; and measure the head movement of the driver after the predetermined time period based on the correct posture characteristic of the driver.

The application may be further configured to: measure an EEG characteristic of the driver using the EEG signal measured during a predetermined time period while the driver is wearing the wearable device and driving the moving device; and measure the EEG signal after the predetermined time period based on the EEG characteristic of the driver.

The wearable device may include: a measurer configured to be worn on an ear of the driver; and a functional member including a battery configured to supply power to the measurer, wherein the functional member may be configured to be fixed to a body part other than the ear of the driver or to a fixed object.

In another general aspect of the disclosure, a driver assistance method, includes: measuring an electroencephalogram (EEG) signal of a driver of a moving device, head movement of the driver, and a traveling information signal of the moving device; processing and providing to a server the measured EEG signal, information about the measured head movement, and the measured traveling information signal; receiving big data-based inattentiveness alarm information from the server based on information provided to the server; providing an inattentiveness-related alarm to the driver; and controlling the moving device based on the inattentiveness alarm information.

The information about the head movement may include at least one of a difference between a head angle of the driver and a reference angle, the number of changes in the head angle of the driver, a duration of a changed head angle of the driver, or any combination thereof.

The inattentiveness alarm information may include at least one of information about a posture of the driver, information about a drowsy state of the driver, or any combination thereof.

The providing of the alarm may further include providing at least one of the inattentiveness-related alarm to the driver according to one or more of the posture, the drowsy state related to the inattentiveness alarm information, or any combination thereof.

The measuring of the EEG signal may further include: measuring a correct posture characteristic of the driver using the information about the head movement measured during a predetermined time period while the driver wears a wearable device and drives the moving device; and measuring the head movement of the driver after the predetermined time period based on the correct posture characteristic of the driver.

The measuring of the EEG signal may further include: measuring an EEG characteristic of the driver using the EEG signal measured during a predetermined time period while the driver is wearing a wearable device and driving the moving device; and measuring the EEG signal after the predetermined time period based on the EEG characteristic of the driver.

In yet another general aspect of the disclosure, a driver assistance system includes: a vehicle configured to gather traveling information; a wearable device configured to measure an electroencephalogram (EEG) signal and head movement of a driver of the vehicle; a mobile terminal including a controller configured to: receive the EEG signal measured by the wearable device; receive information about the head movement measured by the wearable device; and receive traveling information from the vehicle; a server configured to: receive from the mobile terminal, the EEG signal, the information about the head movement, and the traveling information; and generate big data-based inattentiveness alarm information based on information provided to the server by the mobile terminal; and a control module configured to: provide an inattentiveness-related alarm to the driver; and control the vehicle based on the inattentiveness alarm information.

The inattentiveness alarm information may include at least one of information determined based on at least one of traveling information obtained by a plurality of vehicles, the EEG signal for each piece of the traveling information, the information about the head movement for each piece of the traveling information, or any combination thereof.

The information about the head movement may include at least one of a difference between a head angle of the driver and a reference angle, the number of changes in the head angle of the driver, a duration of a changed head angle of the driver, or any combination thereof.

The inattentiveness alarm information may include at least one of information about a posture of the driver, information about a drowsy state of the driver, or any combination thereof.

The control module may be further configured to provide at least one of the inattentiveness-related alarm to the driver according to one or more of the posture, the drowsy state related to the inattentiveness alarm information, or any combination thereof.

The controller may be further configured to: measure a correct posture characteristic of the driver using the information about the head movement measured during a predetermined time period while the driver wears the wearable device and drives the vehicle; and measure the head movement of the driver after the predetermined time period based on the correct posture characteristic of the driver.

Further, the present disclosure provides necessary alarm information for each situation by collecting/refining, by a server end, an electroencephalogram signal for each piece of acquired traveling information and head angle information for each piece of the traveling information and providing an inattentiveness-related alarm.

Furthermore, the present disclosure provides a wearable device with improved performance and light weight by complementing shortcomings of wearable devices used for electroencephalogram measurement.

The effects achievable with the present disclosure are not limited to what has been particularly described hereinabove and other advantages not described herein will be more clearly understood by persons skilled in the art from the following detailed description of the present disclosure.

### BRIEF DESCRIPTION OF THE DRAWINGS

The accompanying drawings, which are included to provide a further understanding of the disclosure and are incorporated in and constitute a part of this application, illustrate embodiment(s) of the disclosure and together with the description serve to explain the principle of the disclosure. In the drawings:

FIG. 1 is a diagram illustrating the configuration of a driver assistance system according to one embodiment of the present disclosure;

FIG. 2 is a diagram illustrating a driver assistance method of a server according to an embodiment of the present disclosure;

FIG. 3 is a diagram showing an example configuration of an entire driver assistance system including a server according to an embodiment of the present disclosure;

FIGS. 4 and 5 are diagrams for illustrating a configuration of a wearable device according to embodiments of the present disclosure;

FIG. 6 is a diagram illustrating a driver assistance method according to an embodiment of the present disclosure;

FIGS. 7 to 9 are diagrams illustrating a method of processing and utilizing head angle information according to an embodiment of the present disclosure;

FIGS. 10 and 11 are diagrams illustrating a method for processing and utilizing an electroencephalogram signal according to an embodiment of the present disclosure;

FIGS. 12 and 13 are diagrams illustrating a driver assistance method according to an embodiment of the present disclosure; and

FIG. 14 is a diagram illustrating the concept of providing an inattentiveness alarm to a driver according to an embodiment of the present disclosure.

### DETAILED DESCRIPTION

Description will now be given in detail according to exemplary embodiments disclosed herein, with reference to the accompanying drawings. The same or equivalent components may be provided with the same reference numbers, and description thereof will not be repeated. As used herein, the suffixes “module” and “part” are added or used interchangeably to facilitate preparation of this specification and are not intended to suggest distinct meanings or functions. In describing embodiments disclosed in this specification, relevant well-known technologies may not be described in detail in order not to obscure the subject matter of the embodiments disclosed in this specification. In addition, it should be noted that the accompanying drawings are only for easy understanding of the embodiments disclosed in the present specification, and should not be construed as limiting the technical spirit disclosed in the present specification. As such, the present disclosure should be construed to extend to any alterations, equivalents and substitutes in addition to those which are particularly set out in the accompanying drawings.

Although the terms first, second, etc. may be used herein to describe various elements, these elements should not be limited by these terms. These terms are generally only used to distinguish one element from another.

It will be understood that when an element is referred to as being “connected with” another element, the element can be directly connected with the other element or intervening elements may also be present. In contrast, it will be understood that when an element is referred to as being “directly connected with” another element, there are no intervening elements present.

A singular representation may include a plural representation unless it represents a definitely different meaning from the context.

The terms such as “include” or “have” used herein are intended to indicate that features, numbers, steps, operations, elements, components, or combinations thereof used in the following description exist and it should be thus understood that the possibility of existence or addition of one or more different features, numbers, steps, operations, elements, components, or combinations thereof is not excluded.

In the following description, the term “moving device” is assumed to mean a vehicle operated by a driver, but it is not limited thereto and includes the concept of various moving means through which risk due to the inattentiveness of the driver is capable of occurring.

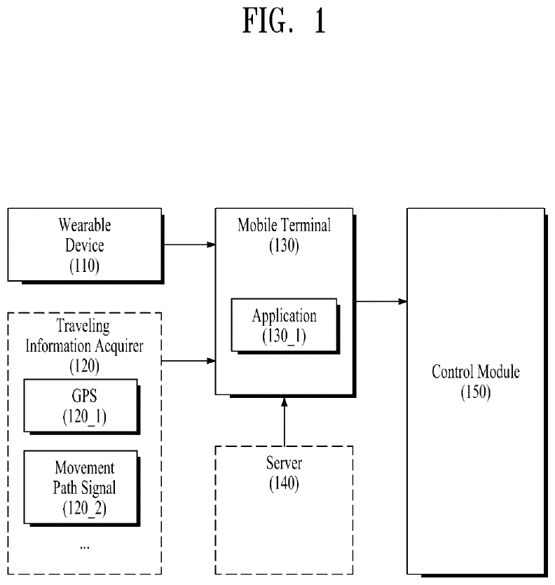

FIG. 1 is a diagram illustrating the configuration of a driver assistance system according to one embodiment of the present disclosure.

Referring to FIG. 1, the driver assistance system according to embodiments may include a wearable device 110 that measures an EEG signal of a driver and a head angle of the driver, and a mobile terminal 130 having an application 130_1, configured to receive the EEG signal measured by the wearable device 110 and information about the head angle measured by the wearable device 110, process the received brain wave signal and the received information about the head angle and provide the same to a server 140, and receive big data-based inattentiveness alarm information. The mobile terminal 130 includes a controller/processor configured to run the application 130_1 in the manner above.

In addition, the driver assistance system according to the present embodiment may include a control module 150 that controls a moving device based on the inattentiveness alarm information received from the server 140 to provide the driver with an inattentive-related alarm.

In addition to the wearable device 110, the mobile terminal 130, and the control module 150, the driver assistance system described above operates by utilizing a traveling information acquirer 120 and a server 140 installed in the moving device as illustrated in FIG. 1. However, the configuration of the traveling information acquirer 120 and the server 140 may not be included in the driver assistance system according to the present embodiment as the configuration of an existing moving device.

As illustrated in FIG. 1, the mobile terminal 130 according to the present embodiment may not only receive and utilize the EEG signal and the head angle information from the wearable device 110, but may also acquire and utilize various traveling information signals from the traveling information acquirer 120. The traveling information signals may include one or more of a GPS signal 120_1, a movement path signal 120_2 of the moving device, a time information signal, and a weather information signal. This information may be acquired from components already equipped in the moving device, or time information, weather information, etc. may be acquired by the mobile terminal 130 itself.

It is proposed that the application 130_1 of the mobile terminal 130 according to the present embodiment utilize inattentiveness alarm information based on big data-based information through the server 140 rather than collecting and processing such traveling information and generating the inattentiveness alarm information by itself. That is, the server 140 may provide more accurate and diverse alarm information by utilizing traveling information corresponding to a plurality of moving devices, and EEG information and head angle information corresponding to the traveling information.

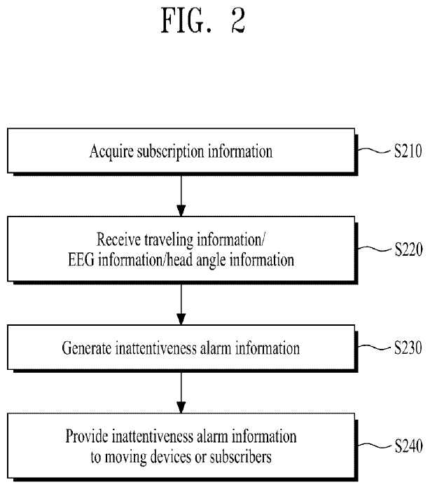

FIG. 2 is a diagram illustrating a driver assistance method of a server according to an embodiment of the present disclosure.

The driver assistance method illustrated in FIG. 2 represents a method performed by the server 140 illustrated in FIG. 1 as an example for providing an assistance service to a bus driver.

First, the server 140 may acquire subscription information of drivers of a plurality of moving devices (S210). The subscription information of the drivers may include, for example, information about a bus transportation company to subscribe to a driver assistance service and subscription information of individual drivers.

Next, the server 140 may receive traveling information, EEG information of each driver of the moving devices, and head angle information of each driver of the moving devices while the moving devices are traveling (S220). The traveling information may include GPS information, movement path information (e.g., bus operation route information, current operation status information, etc.) as described above and the server 140 may receive EEG information according to each movement path. The EEG information and head angle information received by the server 140 may be EEG information or head angle information processed for each driver in the application 130_1 of the mobile terminal 130, and processing of an EEG signal for each individual driver and processing of a head angle for each individual driver will be described in detail below.

The server 140 according to the present embodiment may generate big data-based inattentiveness alarm information based on the traveling information and the EEG information received as described above (S230) and may provide the generated inattentiveness alarm information to the plurality of moving devices or subscribers (S240).

The inattentiveness alarm information may include one or more of information about a movement path or location at which inattentiveness occurs, information about a time period during which inattentiveness occurs, and information about weather under which inattentiveness occurs. Such information is generated at the server 140 end based on information about the plurality of moving devices, rather than at the individual mobile terminal 130 level or an individual moving device level, thereby ensuring objectivity and providing an inattentiveness-related alarm from more diverse aspects.

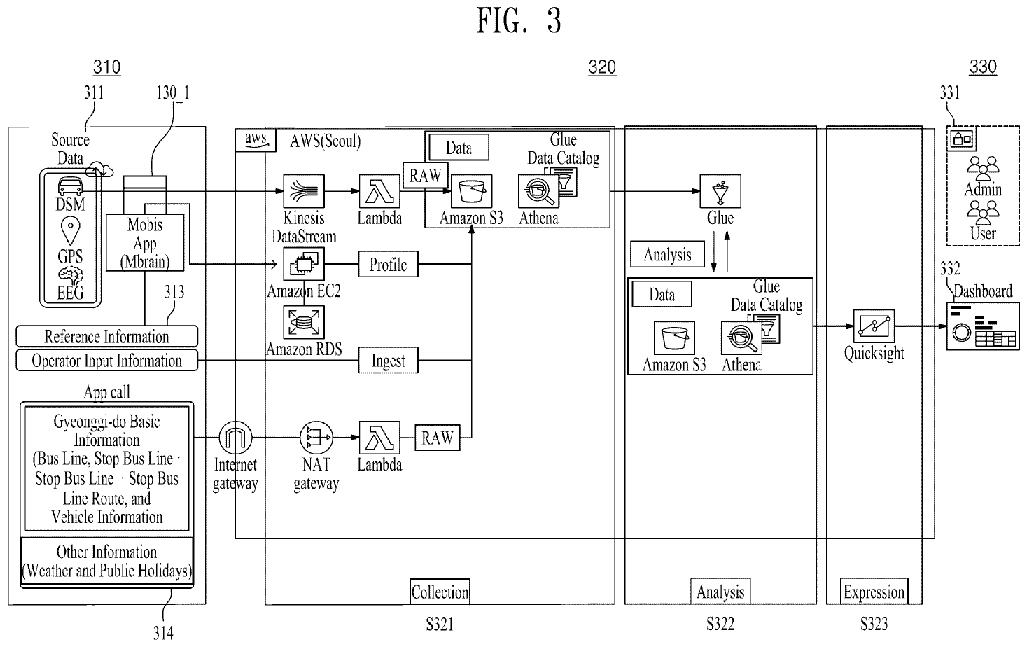

FIG. 3 is a diagram showing an example configuration of an entire driver assistance system including a server according to an embodiment of the present disclosure.

First, in the example of configuration of the system shown in FIG. 3, components are expressed as being divided into an information collection end 310, a server end 320, and a user end 330.

The information collection end 310 may include deep learning-based driver state monitoring (DSM) information, GPS information, and electroencephalogram (EEG) information as source data 311.

In addition, the information collection end 310 may include information acquired via the app 130_1, which may include driver information of the bus company and input information 313 of each driver. However, the driver information of the bus company and the input information 313 of each driver do not necessarily need to be input via the app 130_1 of the mobile terminal 130, and the app 130_1 is able to be installed on various computing devices to provide corresponding reference information.

In addition, the information collection end 310 may include means 314 for acquiring base information (e.g., Gyeonggi-do bus line information, bus line stop information, and vehicle information), weather information, and the like in a corresponding area in addition to the above-described information.

At the server end 320, as described above, the method may be largely divided into collecting the various information from the information collection end 310 (S321), analyzing the information collected as such (S322), and expressing and providing the notification information to the user or a computing device of the bus company using the analyzed result (S323). FIG. 3 shows an example of performing deep learning-based big data conversion of a specific cloud service, but the present embodiment does not need to be limited to such specific cloud service.

The user end 330 may include individual drivers 331 and a computing device dashboard of a bus company 332, as shown in FIG. 3. In the present embodiment, the carelessness notification based on the electroencephalogram-based analysis result may be provided to the individual drivers 331 via various components (e.g., an alarm, a vibration, a display, a sound, and the like) of the mobility. In addition, statistics such as which section of which line the carelessness occurs, carelessness of which driver is frequent, and the like may be provided via the computing device dashboard of the bus company 332.

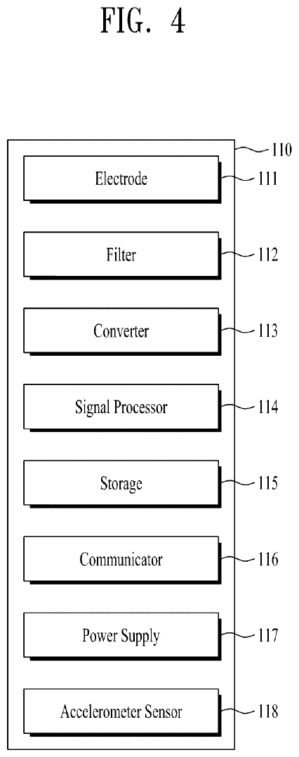

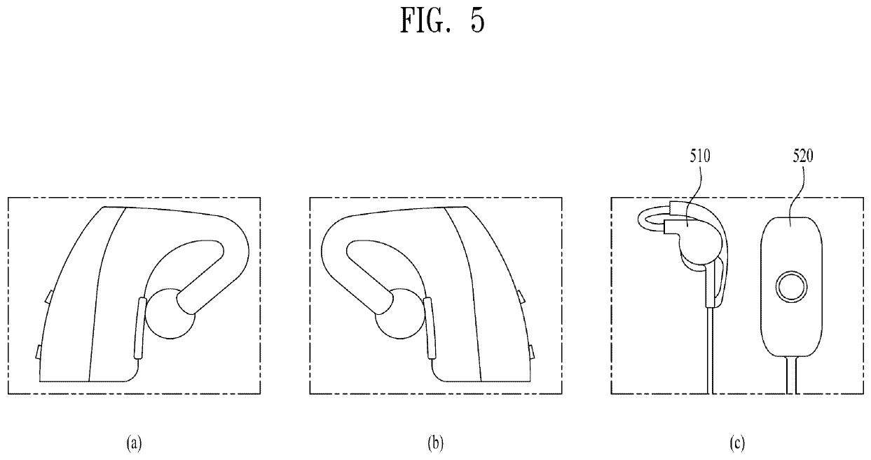

FIGS. 4 and 5 are diagrams for illustrating a configuration of a wearable device according to embodiments of the present disclosure.

The driver assistance system according to one embodiment of the present disclosure may measure the electroencephalogram signal of the driver via the wearable device 110.

The electroencephalogram (EEG) is the phenomenon in which the thoughts, the emotions, and the behaviors of the individual are generated by the communication between the nerve cells in the brain, and is the synchronized electrical wave generated when the nerve cells in the cerebral cortex transmit the signals to each other. As described above, the EEG appears in the various frequency bands, and is able to be specifically classified into the delta wave, the theta wave, the alpha wave, the beta wave, the gamma wave, and the like based on the frequency bands.

In this regard, the wearable device 110 may include the earset that is mounted around the ear of the driver and is able to measure the EEG signal around the left or right temporal lobe of the driver while driving. In another embodiment, the wearable device 110 may include the headset using the multi-channel electrode.

In addition, in the driver assistance system 100 according to one embodiment of the present disclosure, the wearable device 110 may include the various sensors or equipment that may measure the EEG signal of the driver in real time, including the earset and the headset described above.

The electrode 111 may measure the EEG signal of the driver during the driving and/or the stopping of the driver in the vehicle 140 via the electrode. In this regard, the electrode 111 may include the EEG electrode, the REF electrode, and the GND electrode. The EEG electrode is the conductive EEG electrode, and is able to measure the EEG signal of the left or right temporal lobe by being in contact with the area near the left or right temporal lobe temple of the driver. The REF electrode is the reference electrode, and is able to provide the reference potential when the EEG electrode measures the EEG signal. The GND electrode is the ground electrode, is located on the back of the earflap, and is able to serve as the ground when the EEG electrode measures the EEG signal.

In one embodiment, the filter 112 may remove the noise from the EEG signal of the driver measured by the electrode 111. The filter 112 may remove the noise from the EEG signal using the high pass filter, the low pass filter, or the band pass filter. As another embodiment, the filter such as the notch filter or the active filter may be used. In this regard, the noise may include the noise generated by the eye-blink of the driver, the AC power noise, and the DC drift component as well as all the elements that interfere with the feature point extraction.

The converter 113 may convert the noise-removed EEG signal of the driver into the digital signal. The signal processor 114 may calculate one or more characteristics of the EEG from the EEG signal of the driver measured by the wearable device 110. The characteristics of the EEG calculated by the signal processor 114 may be, for example, at least one of the magnitude of the EEG signal in the time domain, the magnitude of the EEG signal in the frequency domain, and the magnitude of the signal-to-noise ratio (SNR) peak.

The storage 115 may store the reference EEG signal, that is, the preset alertness value, for determining the careless state of the driver based on the characteristics of the EEG. In this regard, the careless state of the driver may include the state in which the driver is not able to concentrate on the driving, including the abnormal states such as the drowsiness, the reduced concentration, and the surprise. In addition, the storage 115 may store the EEG signal of the driver measured via the wearable device 110 for a certain period of time. Such storage 115 may include the internal memory and/or the external memory, and may include the volatile memory such as the DRAM, the SRAM, or the SDRAM, the non-volatile memory such as the one time programmable ROM (OTPROM), the PROM, the EPROM, the EEPROM, the mask ROM, the flash ROM, the NAND flash memory, or the NOR flash memory, and the storage device such as the SSD, the compact flash (CF) card, the SD card, the micro-SD card, the mini-SD card, the XD card, the flash drive such as the memory stick, or the HDD.

The communicator 116 is the medium for transmitting and receiving the information between the wearable device 110 and the mobile terminal or the vehicle, and is able to transmit the EEG signal of the driver measured by the wearable device 110 to the mobile terminal or the vehicle via the wireless communication. In the present embodiment, the communicator 116 may transmit and receive the data with the mobile terminal or the vehicle via at least one communication among the Bluetooth, the radio frequency identification (RFID), the infrared data association (IrDA), the ultra-wideband (UWB), the ZigBee, and the wireless fidelity (Wi-Fi) technologies.

The power supply 117 may supply the corresponding power throughout the wearable device 110. Specifically, the power supply 117 may include the converter that converts the AC power to the DC power and the DC/Dc converter that converts the level of the DC power. The power supply 117 may use the scheme of supplying the AC power via the direct connection to the external power source, and may include the power supply 117 that includes the battery to be charged and used.

In the case of the former, the cable is connected to the power supply 117, so that it is difficult for the power supply 117 to move or the range of movement thereof is limited. In the case of the latter, the power supply 117 is free to move, but the weight and the volume thereof increase as much as those of the battery, and the power supply 117 must be directly connected to the power cable or coupled to the charging holder that supplies power for a certain period of time for the charging. The charging holder may be connected to the wearable device 110 via the terminal thereof exposed to the outside, or the built-in battery may be charged when approaching the charging holder using the wireless scheme.

In addition, in the driver assistance system 100 according to the embodiment of the present disclosure, the wearable device 110 may include an accelerometer sensor 118 capable of measuring a head angle of the driver in real time, in addition to the earset and the headset described above.

The accelerometer sensor 118 is a type of motion accelerometer sensor and may sense a change in the axis of a gravitational acceleration direction. That is, the accelerometer sensor 118 may measure the head angle of the driver, more specifically, the angle of an ear and the cervical spine of the driver in a state in which the driver is wearing the wearable device 110.

The storage 115 may store a reference head angle, i.e., a preset reference angle, that may determine the inattentive state of the driver according to a correct posture characteristic. Here, the inattentive state of the driver may include a state in which the driver cannot concentrate on driving, including an abnormal state such as drowsiness, decreased concentration, surprise, etc. and a poor posture state of the driver such as occurrence of a turtle neck posture of the driver during normal driving. In addition, the storage 115 may store, for a certain period of time, the head angle information of the driver obtained through measurement by the wearable device 110. Such storage 115 may include the internal memory and/or the external memory, and may include the volatile memory such as the DRAM, the SRAM, or the SDRAM, the non-volatile memory such as the one time programmable ROM (OTPROM), the PROM, the EPROM, the EEPROM, the mask ROM, the flash ROM, the NAND flash memory, or the NOR flash memory, and the storage device such as the SSD, the compact flash (CF) card, the SD card, the micro-SD card, the mini-SD card, the XD card, the flash drive such as the memory stick, or the HDD.

FIG. 5 illustrates three types of wearable device 110 according to embodiments of the present disclosure.

First, (A) in FIG. 5 shows an example that may be worn on a right ear of the driver, and (B) in FIG. 5 shows an example that may be worn on a left ear of the driver.

In a case of a bus driver in a country where the driver's seat is generally located on a left side, such as Korea, when the driver is wearing the wearable device 110 for measuring the EEG on the right ear as shown in (A) in FIG. 5, there may be a difficulty recognizing a situation in which a passenger approaches and talks. Therefore, in the country such as Korea where the driver's seat is located on the left side, the wearable device 110 that may be worn on the left ear of the user, as shown in (B) in FIG. 5, may be more advantageous.

However, the above description applies to the country where the driver's seat is located on the left side, such as Korea. On the other hand, in countries where the driver's seat is located on a right side, such as Japan/England, the wearable device that is worn on the right ear of the driver as shown in (A) in FIG. 5 may be more advantageous.

On the other hand, the wearable device 110 according to the embodiment shown in (A) and (B) in FIG. 5 may include all of the components described above in FIG. 4, so that the wearable device 110 may be heavy and uncomfortable for the drivers.

Therefore, in a preferred embodiment of the present disclosure, as shown in (C) in FIG. 5, a measurer 510 that may be worn on the ear of the driver and a functional member 520 including a battery that supplies power to the measurer 510 are separated from each other. It is proposed that the functional member 520 is fixed to another body part other than the ear of the driver or a fixture.

In other words, the functional member 520 is constructed to include a component that is heavy and does not necessarily need to be in contact with the driver among the components of the wearable device 110, and to be fixed to clothes of the driver in a form of a clip, thereby reducing the weight of the wearable device 110 while maintaining high performance of the wearable device 110.

For example, the measurer 510 may be configured to include the electrode 111, the filter 112, the converter 113, the signal processor 114, and the accelerometer sensor 118 among the components described above in FIG. 4, and the functional member 520 may be configured to include the storage 115, the communicator 116, and the power supply 117. However, these configurations may be arranged in different ways by comparing advantages when the configurations are connected to the body of the driver with advantages when the configurations are lightweight.

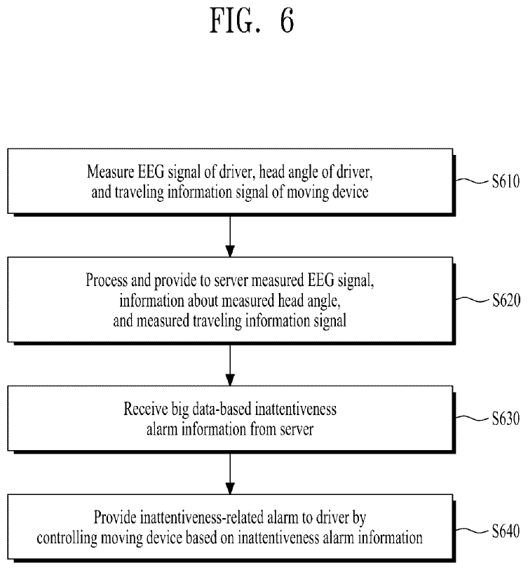

FIG. 6 is a diagram illustrating a driver assistance method according to an embodiment of the present disclosure.

FIG. 6 illustrates a method in which the driver assistance system of FIG. 1 assists the driver based on the application 130_1, the control module 150, etc. illustrated in FIG. 1.

Referring to FIG. 6, the driver assistance method may include measuring an EEG signal of a driver, a head angle of the driver, and a traveling information signal of a moving device (S610), processing the measured EEG signal, information about the measured head angle, and the measured traveling information signal to provide the same to the server (S620), receiving big data-based inattentiveness alarm information from the server (S630), and providing an inattentive-related alarm to the driver by controlling the moving device based on the inattentiveness alarm information (S640).

The measuring step (S610) may be performed by the wearable device 110 of the driver assistance system and the moving device, the providing step (S620) and the inattentiveness alarm information receiving step (S630) may be performed by the application 130_1 of the driver assistance system, and the providing step (S640) may be performed by the control module 150 of the driver assistance system.

Hereinafter, the driver assistance system and the driver assistance method will be described in detail.

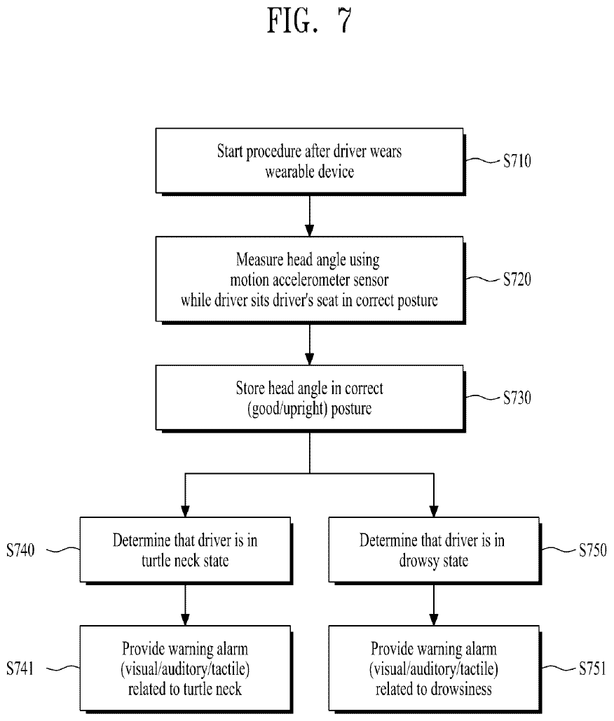

FIGS. 7 to 9 are diagrams illustrating a method of processing and utilizing head angle information according to an embodiment of the present disclosure.

Referring to FIG. 7, first, the driver assistance system and method according to the embodiments may be started when a driver wears the wearable device 110 (S710). When the driver wears the wearable device 110, a head angle of the driver may be measured while the driver sits in a driver's seat (S720). Step S720 may be performed by the wearable device 110 of the driver assistance system and may correspond to step S610 of the driver assistance method.

Then, based on the head angle measured in step S720, information about a correct posture (good/upright posture) of the driver, i.e., the head angle of the driver when the driver is in the correct posture, may be stored in the server 140 (S730). Step S730 may be performed by the wearable device 110 and the application 130_1 of the driver assistance system and may correspond to step S620 of the driver assistance method.

Based on the head angle information in the correct posture stored in step S730, determination is made as to whether the driver is currently driving in a turtle neck state (S740) or whether the driver is currently driving in a drowsy state (S750) based on current head angle information of the driver. In addition, if it is determined that the driver is currently driving in a turtle neck state, a warning alarm related to a turtle neck may be provided to the driver (S741). Alternatively, if it is determined that the driver is currently driving in a drowsy state, a warning alarm related to drowsiness may be provided to the driver (S751). In this case, the warning alarms provided in steps S741 and S751 may include a visual, auditory, and/or tactile alarm.

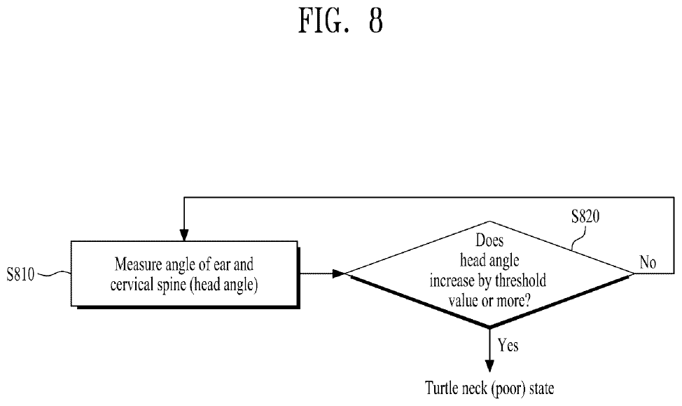

First, referring to FIG. 8 as well, the driver assistance system and method according to embodiments may measure the head angle of the driver, more specifically, the angle of the ear and cervical spine of the driver (S810). Step S810 may be performed by the wearable device 110 of the driver assistance system and may correspond to step S610 of the driver assistance method.

If the head angle detected in step S810 increases by a preset threshold value or more compared to the head angle of the driver in the correct posture stored in step S730, it is determined that the driver is currently driving in a turtle neck state, that is, in a poor posture (S820), and an alarm related to the poor state may be provided to the driver as in step S741. The preset threshold value may be 30 degrees.

The alarm related to the poor state provided in step S741 may be changed according to the degree of a change in the head angle of the driver. For example, if the head angle of the driver increases by a value greater than the preset threshold value compared to the head angle in the correct posture, a stronger alarm may be provided to the driver. Alternatively, for example, if the head angle of the driver increases by a value less than the preset threshold value compared to the head angle in the correct posture, a weaker alarm may be provided to the driver.

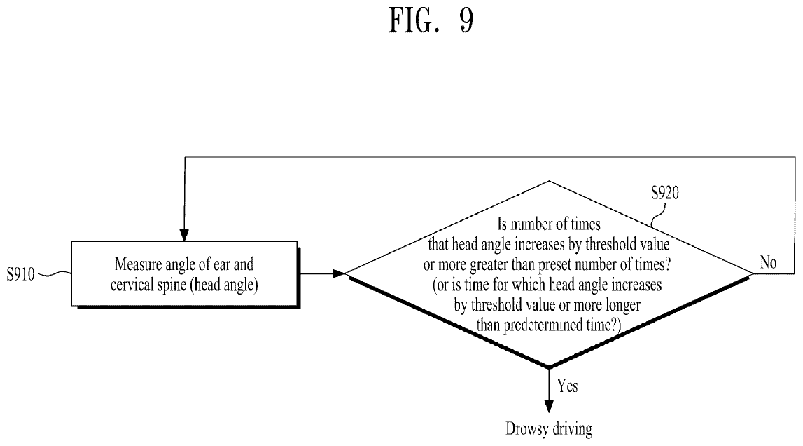

Referring to FIG. 9 as well, the driver assistance system and method according to embodiments may measure the head angle of the driver and, more specifically, the angle of the ear and cervical spine of the driver (S910). Step S910 may be performed by the wearable device 110 of the driver assistance system and may correspond to step S610 of the driver assistance method.

If the number of times that the head angle of the driver detected in step S910 increases by the preset threshold value or more compared to the head angle of the driver in the correct posture stored in step S730 is greater than a preset number of times or if a time for which the head angle increases by the preset threshold value or more is longer than a predetermined time, it is determined that the driver is currently driving in a drowsy state (S920) and an alarm related to drowsiness may be provided to the driver as in step S751. The preset threshold value may be 30 degrees.

For example, in step S920, if it is determined that the number of times that the head angle of the driver detected in step S910 increases by the preset threshold value or more compared to the head angle of the driver in the correct posture stored in step S730 is three or more times within 10 seconds, it may be determined that the driver is currently driving in a drowsy state and provide the driver with the alarm related to drowsiness as in step S751.

Alternatively, for example, in step S920, if it is determined that the time for which the head angle of the driver detected in step S910 increases by the preset threshold value or more compared to the head angle of the driver in the correct posture stored in step S730 is 3 seconds or more, it may be determined that the driver is currently driving in a drowsy state and the alarm related to drowsiness may be provided to the driver as in step S751.

The alarm related to drowsiness provided in step S751 may be changed according to the number of changes in the head angle of the driver. For example, if the number of times that the head angle of the driver increases by the preset threshold value or more compared to the head angle in the correct posture increases by a value greater than the preset number of times, a stronger alarm may be provided to the driver. Alternatively, for example, if the number of times that the head angle of the driver increases by the preset threshold value or more compared to the head angle in the correct posture increases by a value less than the preset number of times, a weaker alarm may be provided to the driver.

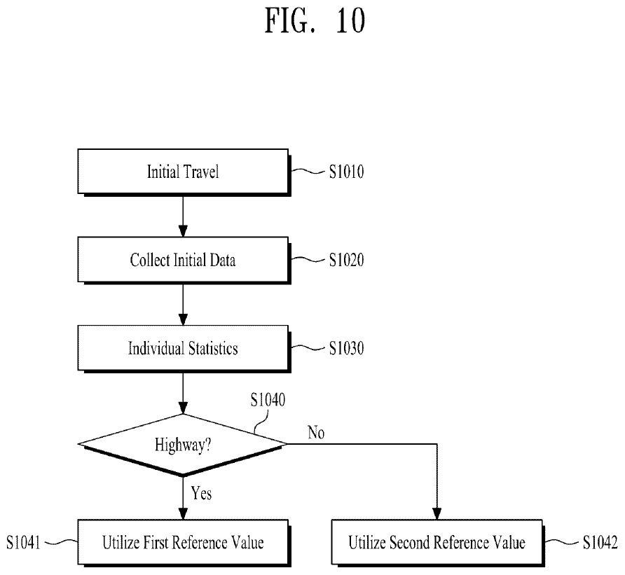

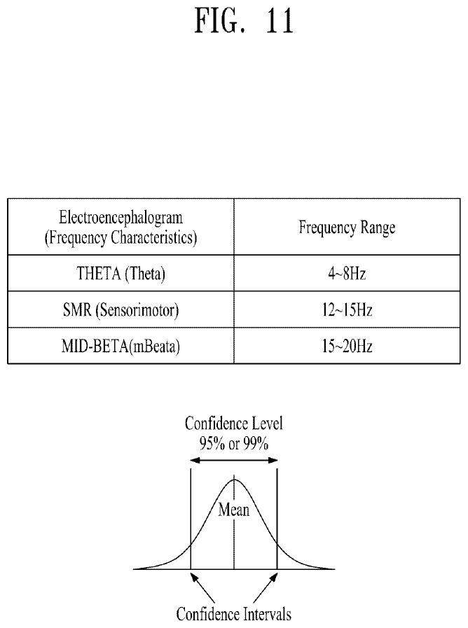

FIGS. 10 and 11 are diagrams for illustrating a method for processing and utilizing an EEG signal according to an embodiment of the present disclosure.

For measuring the EEG signal of the driver via the wearable device 110, measuring the EEG of the driver for each waveform (e.g., theta, SMR, and mid-beta in frequency ranges indicated at top of FIG. 11) on the same standard and quantifying the measurement results may be considered, but this may not take into account the differences in the EEG signals depending on the alertness between the drivers. Therefore, in a preferred embodiment of the present disclosure, it is proposed to consider the EEG characteristics of each driver and determine a degree of carelessness based on the characteristics.

To this end, in the embodiment shown in FIG. 10, first, the EEG signal may be measured during a predetermined time period while the driver wears the wearable device 110 and drives (S1010).

The predetermined time period is a time period for extracting the EEG characteristics for each driver using the EEG during initial travel of the driver, and is assumed to be set to 5 minutes. After collecting EEG data of the driver for the first 5 minutes as such (S1020), statistics on carelessness numeric values of the corresponding individual driver may be calculated based on the collected data (S1030). For example, such statistics on the carelessness numeric values of each individual may be calculated as a distribution as shown at bottom of FIG. 11. Mean (μ) and standard deviation (σ) may be calculated from the distribution, and may be recorded/saved. In this regard, it may be efficient to measure the EEG signal by excluding an alertness score with a deviation equal to or greater than 3σ as an outlier.

When the degree of carelessness of each driver is determined using the mean (μ) and the standard deviation (σ) as described above, an alarm may be provided on the degree of carelessness of the corresponding driver in consideration of individual characteristics of the driver.

In the preferred embodiment of the present disclosure, it is proposed to additionally consider a travel state in determining the degree of carelessness of the driver. As an example, in FIG. 10, it is proposed to provide the carelessness alarm based on different standards by determining whether the corresponding mobility is travelling on a highway (S1040). Whether the mobility is traveling on the highway may be determined by whether the corresponding mobility maintains a travel at a speed equal to or higher than 60 km/h for 10 seconds.

When the corresponding mobility is traveling on the highway, whether to provide the carelessness alarm may be determined based on a first reference value (S1050). For example, when the mobility is traveling on the highway, and when the alertness score is equal to or smaller than μ-1.96σ (the first reference value), it may be set to provide the carelessness alarm to the corresponding driver, which may correspond to a 95% confidence interval in the distribution shown at the bottom of FIG. 11.

On the other hand, when the mobility is traveling on a general road, and when the alertness score is equal to or smaller than μ-2.58σ (a second reference value), it may be set to provide the carelessness alarm to the corresponding driver, which may correspond to 99% confidence interval in the distribution shown at the bottom of FIG. 11. In other words, in the present embodiment, it is proposed to prevent major accidents in advance by providing the carelessness alarm using a standard higher than that in the case of traveling on the highway.

In the above-described embodiment, it is desirable to set the carelessness alarm not to be provided for 5 minutes set to identify the EEG characteristics of each driver at start of each travel. Additionally, when the driver is not properly wearing the wearable device, a separate notification may be provided, but it is desirable to set the carelessness alarm not to be activated in such situation.

Although the example of recording/saving the EEG characteristics of the specific driver for 5 minutes after the start of travel is illustrated in the above-described embodiment, the EEG characteristics of the driver may also be used in a scheme of being managed by the server by calculating the mean and the standard deviation in a 24-hour period.

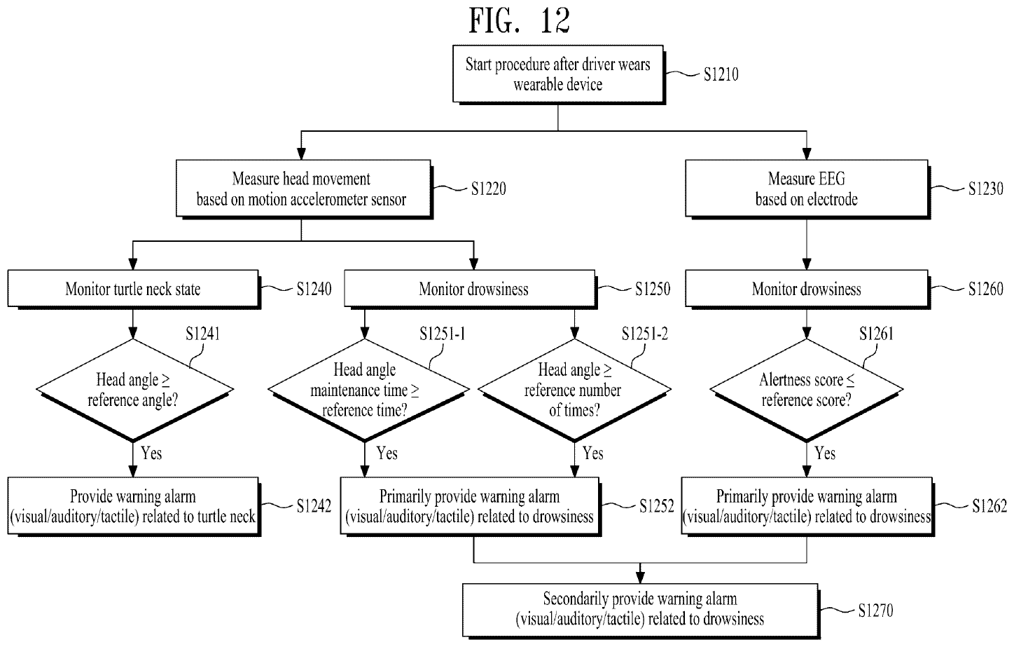

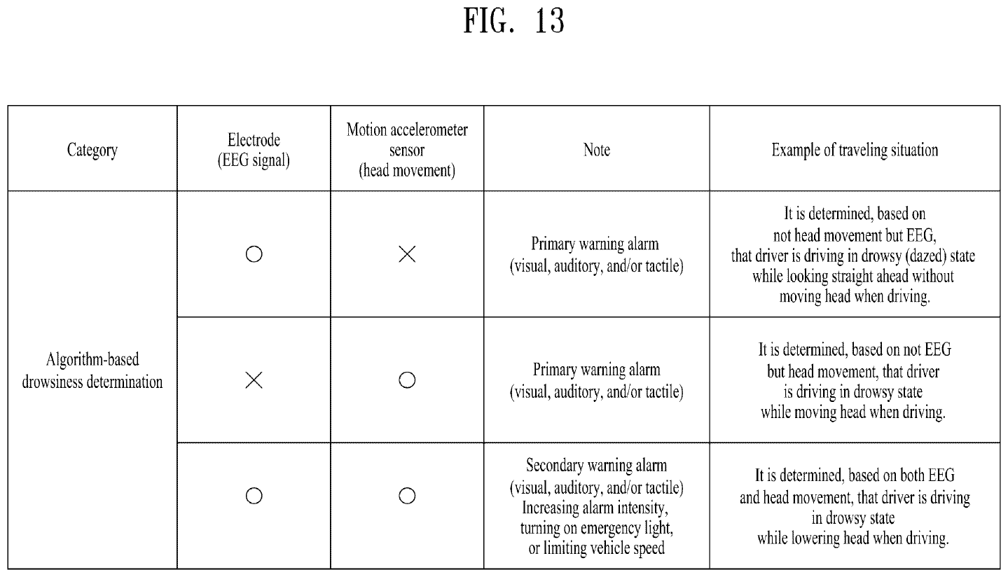

FIGS. 12 and 13 are diagrams illustrating a driver assistance method according to an embodiment of the present disclosure.

Referring to FIG. 12, the driver assistance system and method according to the embodiments may be started when a driver wears the wearable device 110 (S1210). Step S1210 may correspond to step S710 of the driver assistance method. When the driver wears the wearable device 110, a head angle of the driver while the driver is sitting in a driver's seat may be measured (S1220) or an EEG of the driver may be measured (S1230).

Based on the head angle of the driver measured in step S1220, it may be determined whether the driver is currently driving in a turtle neck state (S1240) or whether the driver is currently driving in a drowsy state (S1250).

For example, as described in FIG. 8, the driver assistance system and method according to the embodiments may determine that the driver is currently driving in a turtle neck state, i.e., a poor posture, if the measured head angle of the driver increases by the preset threshold value or more compared to the head angle of the driver in the correct posture stored in step S730 (selection of Yes in step S1241). In this case, an alarm related to the poor state may be provided to the driver as in step S741 (S1242). In this case, the warning alarm provided in step S1242 may include a visual, auditory, and/or tactile alarm.

Alternatively, as described in FIG. 9, the driver assistance system and method according to the embodiments may determine that the driver is currently driving in a drowsy state if the number of times that the measured head angle of the driver increases by the preset threshold value or more compared to the head angle of the driver in the correct posture stored in step S730 is greater than the preset number of times or if a time for which the head angle increases by the preset threshold value or more is longer than a predetermined time, it is determined that the driver is currently driving in a drowsy state (selection of Yes in step S1251-1 or selection of Yes in step S1251-2). In this case, an alarm related to a drowsy state may be provided to the driver as in step S751. In this case, the warning alarm provided in step S1252 may include the visual, auditory, and/or tactile alarm.

Alternatively, as described in FIGS. 10 and 11, the driver assistance system and method according to the embodiments may determine that the driver is currently driving in a drowsy state if the alertness score determined based on the EEG signal of the driver measured by the wearable device 110 is lower than a preset alertness score stored in the storage 115 (selection of Yes in step S1261). In this case, an alarm related to the drowsy state may be provided to the driver (S1262). The warning alarm provided in step S1262 may include the visual, auditory, and/or tactile alarm.

The driver assistance system and method according to the embodiments may secondarily provide the drowsiness warning alarm to the driver as in step S1270, if it is determined based on the head angle that the driver is currently driving in a drowsy state and, at the same time, if it is determined based on the EEG that the driver is currently driving in a drowsy state in steps S1250 and S1260. In this case, the warning alarm provided in step S1270 may include the visual, auditory, and/or tactile alarm.

Referring to FIG. 13 as well, the driver assistance system and method according to the embodiments may determine whether the driver is currently driving in a drowsy state based on an EEG signal of the driver and the head angle (head movement) of the driver.

For example, according to the embodiments, it may be determined based on the EEG signal of the driver that the driver is currently driving in a drowsy state but it may be determined based on the head movement of the driver that the driver is not currently driving in a drowsy state. Such a case may be, for example, a case in which it is determined, based only on EEG analysis, that the driver is driving in a drowsy (dazed) state while looking straight ahead without moving the head of the driver when driving. In this case, the driver assistance system and method according to the embodiments may primarily provide an alarm related to drowsiness to the driver.

Alternatively, for example, in some embodiments, it may be determined based on the EEG signal of the driver that the driver is not currently driving in a drowsy state but it may be determined based on the head movement of the driver that the driver is currently driving in a drowsy state. For example, this may be the case in which it is determined based on only the head movement that the driver is driving in a drowsy state while moving the head of the driver when driving. In this case, the driver assistance system and method according to the embodiments may primarily provide the driver with an alarm related to drowsiness.

Alternatively, for example, in some embodiments, it may be determined based on the EEG signal of the driver that the driver is currently driving in a drowsy state and, at the same time, it may be determined based on the head movement of the driver that the current driver is driving in a drowsy state. In such a case, it may be determined based on the EEG signal and the head movement that the driver is driving in a drowsy state while lowering the head when driving. In this case, the driver assistance system and method according to the embodiments may secondarily provide the driver with an alarm related to drowsiness. That is, unlike the two cases described above, alarm intensity may be increased or a stronger alarm related to drowsiness may be provided to the driver, such as turning on an emergency light or limiting a vehicle speed.

Therefore, the driver assistance system and method according to the embodiments have the effect of more accurately determining whether the driver is currently driving in a drowsy state by simultaneously considering the EEG signal of the driver and the head movement of the driver.

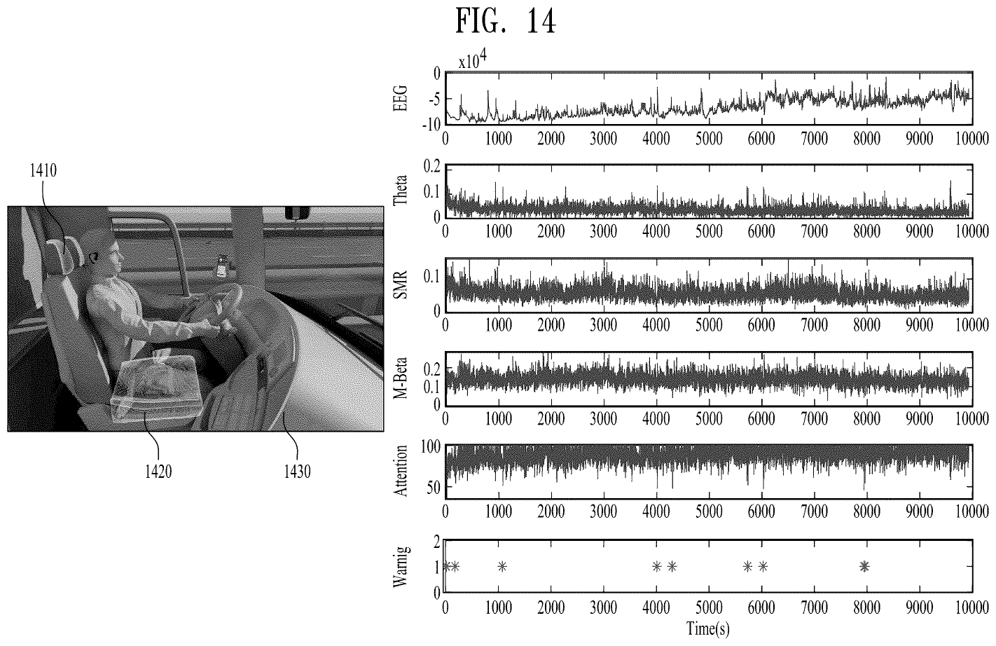

FIG. 14 is a diagram illustrating the concept of providing an inattentiveness alarm to a driver according to an embodiment of the present disclosure.

A left side of FIG. 14 exemplarily illustrates a method of providing an inattentiveness alarm to a driver by providing sound through a speaker 1410 located in a headrest part, a method of providing vibration and/or ventilation through a lower part 1420 of a driver's seat, and a method of providing an inattentiveness alarm by turning on a light 1430 such as an LED placed in front of the driver's seat.

A right side of FIG. 14 illustrates various types of EEG measured through the wearable device 110 worn by the driver. That is, the EEG measured from the driver is not limited to theta, SMR, and mid-beta indicated in an upper portion of FIG. 11, and various types of EEG may be measured.

While the above-described embodiments of the present disclosure have been described based on a method of providing an inattentiveness alarm to a driver based on EEG, in another embodiment of the present disclosure, such an EEG-based inattentiveness alarm method and an image-based inattentiveness alarm method may be used in combination.

The EEG-based inattentiveness alarm method has the advantage of quickly determining an inattentive state of the driver and providing an alarm as compared to the image-based inattentiveness alarm method. For example, the image-based inattentiveness alarm method may provide an alarm related to inattentiveness through an image change of actual eyelid closing in a drowsy situation of the driver, whereas the EEG-based inattentiveness alarm method has the advantage of providing an alarm about the inattentiveness status through the brainwave of the driver before the actual eyelid closing situation.

However, in a situation in which the driver is focused but not looking ahead rather than a situation in which the driver is in a drowsy state, the image-based inattentiveness alarm method may have an advantage over the EEG-based inattentiveness alarm method. Therefore, in an embodiment of the present disclosure, in addition to the above-described inattentiveness alarm based on the EEG of the driver, an alarm for a situation in which the driver neglects to look ahead may be provided based on the image.

In addition, for the convenience of explanation, while the description has been given based on the EEG signal of the driver, it will be apparent that the same is applicable to the inattentiveness alarm method based on the head angle of the driver, i.e. head movement information.

As described above, the detailed description of the embodiments of the present disclosure has been given to enable those skilled in the art to implement and practice the disclosure. Although the disclosure has been described with reference to the embodiments, those skilled in the art will appreciate that various modifications and variations may be made in the present disclosure without departing from the spirit or scope of the disclosure and the appended claims. For example, those skilled in the art may use constructions disclosed in the above-described embodiments in combination with each other.

Accordingly, the present disclosure should not be limited to the specific embodiments described herein, but should be accorded the broadest scope consistent with the principles and features disclosed herein.

What is claimed is:

1. A driver assistance system, comprising:
a wearable device configured to measure an electroencephalogram (EEG) signal and head movement of a driver of a moving device;
a mobile terminal including an application configured to:
receive the EEG signal measured by the wearable device;
receive information about the head movement measured by the wearable device;
receive a traveling information signal acquired by the moving device;
process and provide to a server the received EEG signal, the received information about the head movement, and the received traveling information signal; and
receive big data-based inattentiveness alarm information from the server based on information provided to the server by the mobile terminal; and
a control module configured to:
provide an inattentiveness-related alarm to the driver; and
control the moving device based on the inattentiveness alarm information.

2. The driver assistance system of claim 1, wherein the inattentiveness alarm information includes at least one of information determined based on at least one of traveling information obtained by a plurality of moving devices, the EEG signal for each piece of the traveling information, the information about the head movement for each piece of the traveling information, or any combination thereof.

3. The driver assistance system of claim 1, wherein the information about the head movement includes at least one of a difference between a head angle of the driver and a reference angle, the number of changes in the head angle of the driver, a duration of a changed head angle of the driver, or any combination thereof.

4. The driver assistance system of claim 3, wherein the inattentiveness alarm information includes at least one of information about a posture of the driver, information about a drowsy state of the driver, or any combination thereof.

5. The driver assistance system of claim 4, wherein the control module is further configured to provide at least one of the inattentiveness-related alarm to the driver according to one or more of the posture, the drowsy state related to the inattentiveness alarm information, or any combination thereof.

6. The driver assistance system of claim 1, wherein the application is further configured to:
measure a correct posture characteristic of the driver using the information about the head movement measured during a predetermined time period while the driver wears the wearable device and drives the moving device; and
measure the head movement of the driver after the predetermined time period based on the correct posture characteristic of the driver.

7. The driver assistance system of claim 1, wherein the application is further configured to:
measure an EEG characteristic of the driver using the EEG signal measured during a predetermined time period while the driver is wearing the wearable device and driving the moving device; and
measure the EEG signal after the predetermined time period based on the EEG characteristic of the driver.

8. The driver assistance system of claim 1, wherein the wearable device includes:
a measurer configured to be worn on an ear of the driver; and
a functional member including a battery configured to supply power to the measurer,
wherein the functional member is configured to be fixed to a body part other than the ear of the driver or to a fixed object.

9. A driver assistance method, comprising:
measuring an electroencephalogram (EEG) signal of a driver of a moving device, head movement of the driver, and a traveling information signal of the moving device;
processing and providing to a server the measured EEG signal, information about the measured head movement, and the measured traveling information signal;
receiving big data-based inattentiveness alarm information from the server based on information provided to the server;
providing an inattentiveness-related alarm to the driver; and
controlling the moving device based on the inattentiveness alarm information.

10. The driver assistance method of claim 9, wherein the information about the head movement includes at least one of a difference between a head angle of the driver and a reference angle, the number of changes in the head angle of the driver, a duration of a changed head angle of the driver, or any combination thereof. 11 The driver assistance method of claim 10, wherein the inattentiveness alarm information includes at least one of information about a posture of the driver, information about a drowsy state of the driver, or any combination thereof.

12. The driver assistance method of claim 11, wherein the providing of the alarm further includes providing at least one of the inattentiveness-related alarm to the driver according to one or more of the posture, the drowsy state related to the inattentiveness alarm information, or any combination thereof.

13. The driver assistance method of claim 9, wherein the measuring of the EEG signal further includes:
measuring a correct posture characteristic of the driver using the information about the head movement measured during a predetermined time period while the driver wears a wearable device and drives the moving device; and
measuring the head movement of the driver after the predetermined time period based on the correct posture characteristic of the driver.

14. The driver assistance method of claim 9, wherein the measuring of the EEG signal further includes:
measuring an EEG characteristic of the driver using the EEG signal measured during a predetermined time period while the driver is wearing a wearable device and driving the moving device; and
measuring the EEG signal after the predetermined time period based on the EEG characteristic of the driver.

15. A driver assistance system, comprising:
a vehicle configured to gather traveling information;
a wearable device configured to measure an electroencephalogram (EEG) signal and head movement of a driver of the vehicle;
a mobile terminal including a controller configured to:
receive the EEG signal measured by the wearable device;
receive information about the head movement measured by the wearable device; and
receive the traveling information from the vehicle;
a server configured to:
receive from the mobile terminal, the EEG signal, the information about the head movement, and the traveling information; and
generate big data-based inattentiveness alarm information based on information provided to the server by the mobile terminal; and
a control module configured to:
provide an inattentiveness-related alarm to the driver; and
control the vehicle based on the inattentiveness alarm information.

16. The system of claim 15, wherein the inattentiveness alarm information includes at least one of information determined based on at least one of traveling information obtained by a plurality of vehicles, the EEG signal for each piece of the traveling information, the information about the head movement for each piece of the traveling information, or any combination thereof.

17. The system of claim 15, wherein the information about the head movement includes at least one of a difference between a head angle of the driver and a reference angle, the number of changes in the head angle of the driver, a duration of a changed head angle of the driver, or any combination thereof.

18. The system of claim 17, wherein the inattentiveness alarm information includes at least one of information about a posture of the driver, information about a drowsy state of the driver, or any combination thereof.

19. The driver assistance system of claim 18, wherein the control module is further configured to provide at least one of the inattentiveness-related alarm to the driver according to one or more of the posture, the drowsy state related to the inattentiveness alarm information, or any combination thereof.

20. The driver assistance system of claim 15, wherein the controller is further configured to:
measure a correct posture characteristic of the driver using the information about the head movement measured during a predetermined time period while the driver wears the wearable device and drives the vehicle; and
measure the head movement of the driver after the predetermined time period based on the correct posture characteristic of the driver.
